(19) United States
(12) Patent Application Publication
(10) Pub. No.: US 2023/0268050 A1
(43) Pub. Date: Aug. 24, 2023

(54) Circadian Rhythm Recommendation Model Using Light Sensors and an Intelligent Light Box

(71) Applicant: Serca Science LLC, New Haven, CT (US)

(72) Inventors: Peter Kahn, New Haven, CT (US); Melissa Knauert, Orange, CT (US); Bezhou Feng, Cleveland Heights, OH (US); Vishhvaan Gopalakrishnan, Beachwood, OH (US)

(21) Appl. No.: 18/109,919

(22) Filed: Feb. 15, 2023

Related U.S. Application Data
(60) Provisional application No. 63/312,150, filed on Feb. 21, 2022.

(51) Int. Cl. G16H 20/30; G16H 50/70; G16H 10/60; G16H 80/00; A61B 5/00

(52) U.S. Cl. CPC G16H 20/30; G16H 50/70; G16H 10/60; G16H 80/00; A61B 5/4815; A61B 5/6803; A61B 5/742

(57) ABSTRACT

Aspects of the disclosure relate to circadian rhythm improvement. A computing platform may receive light information from a light sensor device, and user information/desired outcome information from a user device. The computing platform may input, into a model, the light information and the user information, which may cause the model to produce a control signal and a circadian rhythm improvement recommendation. The computing platform may send, to the user device, the circadian rhythm improvement recommendation and commands directing the user device to display the circadian rhythm improvement recommendation, which may cause the user device to display the circadian rhythm improvement recommendation. The computing platform may receive, from the user device, feedback information indicating compliance with the circadian rhythm improvement recommendation, and may refine, using the feedback information, the model.

### CROSS-REFERENCE TO RELATED APPLICATIONS

This application claims priority to U.S. Provisional Patent Application No. 63/312,150, filed Feb. 21, 2022, and entitled “Devices, Systems, and Methods for Optimizing Human Circadian Rhythms,” which is hereby incorporated herein by reference in its entirety.

### TECHNICAL FIELD

The devices, systems, and methods described herein relate generally to assisting users in optimizing their circadian rhythms and, in so doing, optimize their sense of wellbeing, improve their health, and/or other personal goals. More specifically, light sensors, a circadian rhythm analysis and recommendation model, and an intelligent light box are described. Disclosed embodiments include sensors for capturing data on the user's environment (e.g., light exposure) during their daily routine. That data, along with self-reported information about the user, may be used by a computer program to estimate the user's current circadian rhythm and make recommendations to the user regarding lifestyle adjustments that they can make to adjust their circadian rhythm to achieve their desired outcomes (e.g., align with their daily schedule, make adjustments for travel). Other embodiments include devices, such as adjustable “smart” light boxes, to provide users with exposure to light of the recommended color or wavelength, and intensity at the appropriate time for the appropriate duration to maintain or adjust their circadian rhythm according to their preference.

### BACKGROUND

Human circadian rhythms are physical, mental, and behavioral changes that follow a 24-hour cycle, primarily in response to the light and dark cycles that are an effect of the Earth's rotation around its axis and around the Sun. Certain physiological factors typically correlate to circadian rhythms including, for example, body temperature, blood pressure, and melatonin levels. Sleep or circadian medicine involves the science of studying these external and internal factors, and correlating them to improve patient sleep quality, alertness during the day, and even memory processing. Accurate knowledge of one's circadian rhythm also allows individuals to arrange their schedule to take advantage of better times of day for certain types of activities (e.g., mentally-focused activities versus physically-focused activities, accounting for jet-lag, adjusting to night or evening shift work schedules, and/or other activities), manage and/or otherwise improve techniques for weight loss, and/or achieve other benefits.

However, in modern advanced societies, the natural circadian rhythm can be disrupted in ways that are detrimental to an individual's physical and mental health resulting in adverse health outcomes ranging from increased risk of cardiovascular and psychological morbidity to simply lower quality of life. This disruption of the circadian rhythm can be a result of, among other things, exposure to artificial light, use of drugs and alcohol (both legal and illegal), meal times, exercise routine, inconsistency of sleep and wake times, and/or other factors.

There are known treatments to address disrupted circadian rhythms, including use of light exposure via artificial sources of light (such as light boxes) or natural sources (such as the sun), consistency in wake and sleep times, diet and exercise recommendations, use of dietary supplements such as melatonin, scheduling of meals, and limitations on drug and alcohol use. However, absent accurate information on an individual's current daily routine and exposure to environmental factors, it is difficult to recommend appropriate subject-specific treatments (e.g., by algorithms, individuals (e.g., healthcare providers or the like), and/or otherwise). While self-reporting may be adequate for certain types of information (e.g., sleep and wake times), other kinds of information (e.g., exposure to light levels during the day) are ill-suited for self-reporting at a level of accuracy sufficient to be useful or relied upon. Furthermore, even when appropriate treatments can be determined, individuals may lack the self-discipline to maintain (or reliably self-report on) adherence to those recommendations.

### SUMMARY

Aspects of the disclosure provide effective, efficient, scalable, and convenient technical solutions that address and overcome the technical problems associated with circadian rhythm optimization. In accordance with one or more embodiments of the disclosure, a computing platform comprising at least one processor, a communication interface, and memory storing computer-readable instructions may receive, from a light sensor device and via the communication interface, light information. The computing platform may receive, from a user device and via the communication interface, user information and desired outcome information. The computing platform may input, into a circadian rhythm recommendation model, the light information, the desired outcome information, and the user information, which may cause the circadian rhythm recommendation model to produce a first circadian rhythm improvement recommendation. The computing platform may send, to the user device, the first circadian rhythm improvement recommendation and one or more commands directing the user device to display the first circadian rhythm improvement recommendation, which may cause the user device to display the first circadian rhythm improvement recommendation. The computing platform may receive, from the user device, feedback information indicating compliance with the first circadian rhythm improvement recommendation. The computing platform may refine, using the feedback information, the circadian rhythm recommendation model.

In one or more instances, the computing platform may train, using historical information, the circadian rhythm recommendation model, which may configure the circadian rhythm recommendation model to produce control signals and circadian rhythm improvement recommendations. In one or more instances, the historical information may correspond to a plurality of users.

In one or more examples, the historical information may be one or more of: historical light information, historical user input information, or historical biometric information. In one or more examples, the light information may be one or more of: light intensity across a visible spectrum or light intensity outside of the visible spectrum.

In one or more instances, the user information may be one or more of: age, weight, gender, daily schedule, wake and sleep times, circadian phenotype, activity information, menstrual cycle data, nutrition information, metabolic/metabolism data, cardio/pulmonary activity data, location information, or weather information. In one or more instances, the desired outcome information may be one or more of: travel adjustments or schedule adjustments.

In one or more examples, the computing platform may receive biometric information from a biometric sensor, where producing the first circadian rhythm improvement recommendation may be further based on the biometric information. In one or more examples, the biometric information may be one or more of: pulse information, heartrate information, electrocardiogram readings, or movement information.

In one or more instances, the first circadian rhythm improvement recommendation may include one or more of: use of a light box, adjust sun exposure, adjust light exposure, a recommended wake up time, a recommended sleep schedule, or travel advice. In one or more instances, inputting the light information and the user information into the circadian rhythm recommendation model may further cause the circadian rhythm recommendation model to produce a control signal directed to an intelligent light box device.

In one or more examples, the computing platform may send, to the intelligent light box device, the control signal, which may cause the intelligent light box device to perform one or more of: adjust a frequency of data transmission, adjust an intensity of emitted light, adjust a color of emitted light, adjust a time of use, or adjust a duration of use. In one or more examples, the light sensor device may be configured to communicate an observed intensity of light to the intelligent light box device, and the intelligent light box device may be configured to automatically adjust an emitted intensity of light based on the observed intensity of light at the light sensor device.

In one or more instances, the computing platform may send, to the light sensor device, a control signal, which may cause the light sensor device to modify one or more operations of the light sensor device. In one or more instances, modifying the one or more operations of the light sensor device may include one or more of: adjust polling intervals, adjust sleep intervals, or adjust data transmission frequency. In one or more instances, the light sensor device may be mounted to a pair of glasses.

### BRIEF DESCRIPTION OF DRAWINGS

FIGS. 1A-1I depict an illustrative computing environment for implementing light sensors, a circadian rhythm recommendation model, and an intelligent light box in accordance with one or more example embodiments;

FIGS. 2A-2E depict an illustrative event sequence for implementing light sensors, a circadian rhythm recommendation model, and an intelligent light box in accordance with one or more example embodiments;

FIGS. 3-5 depict illustrative methods for implementing light sensors, a circadian rhythm recommendation model, and an intelligent light box in accordance with one or more example embodiments; and

FIGS. 6A-6S depict illustrative graphical user interfaces for implementing light sensors, a circadian rhythm recommendation model, and an intelligent light box in accordance with one or more example embodiments.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. In some instances, other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

As a brief introduction to the concepts described in further detail below, systems and methods for assisting users in optimizing their circadian rhythms (and in so doing optimizing their sense of wellbeing) are described herein. More specifically, light sensors, a circadian rhythm analysis and recommendation model, and an intelligent light box are described. Disclosed embodiments include sensors for capturing data on the user's environment (e.g., light exposure) during their daily routine. That data, along with self-reported information about the user, may be used by a computer program to estimate the user's current circadian rhythm and make recommendations to the user regarding lifestyle adjustments that they can make to more closely align their circadian rhythm with their everyday schedule. Other embodiments include devices, such as adjustable “smart” light boxes, to provide users with exposure to light of the recommended color or wavelength, and intensity at the appropriate time for the appropriate duration to maintain or adjust their circadian rhythm according to their preference.

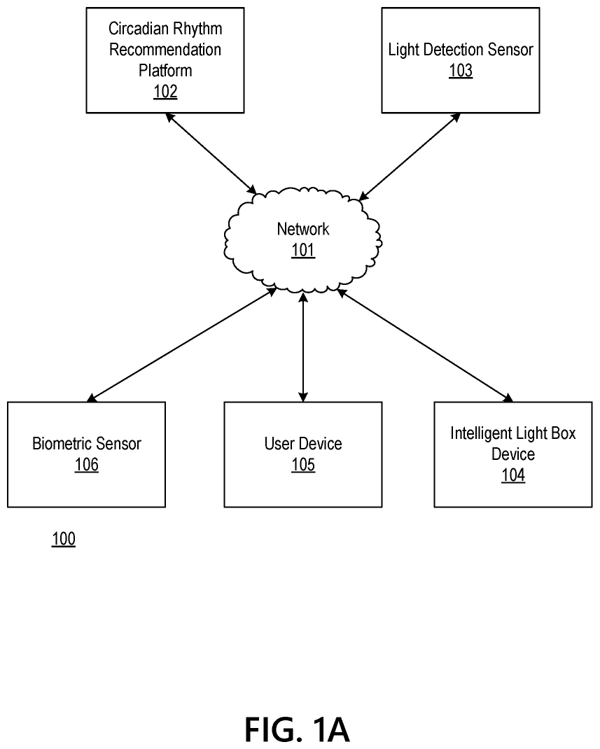

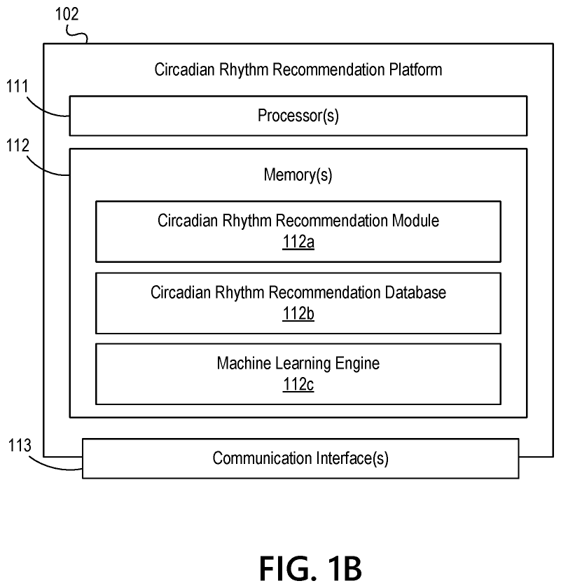

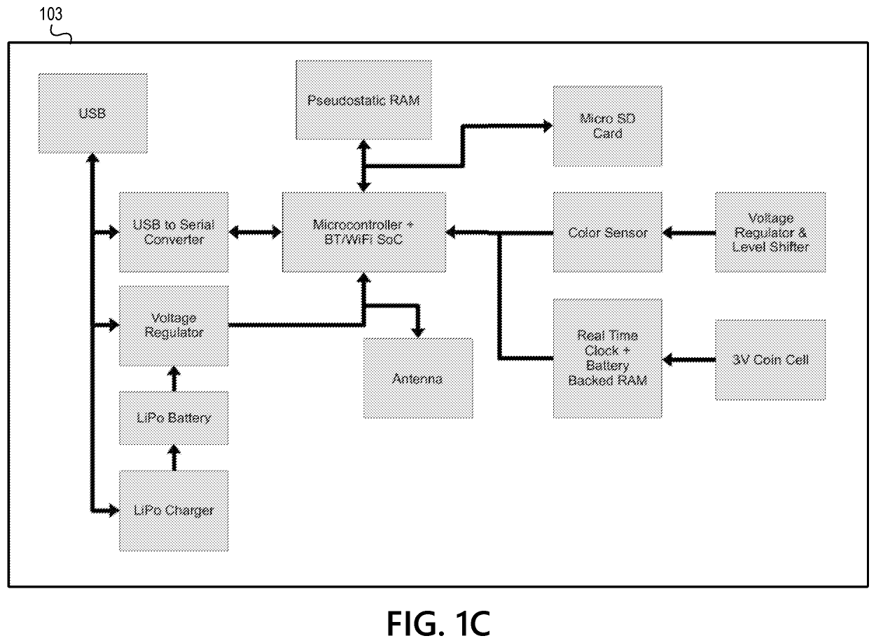

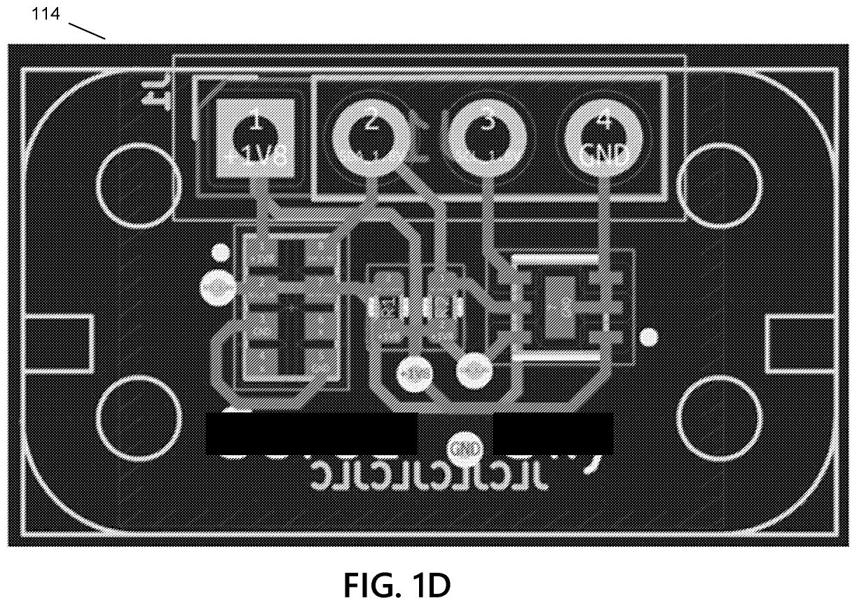

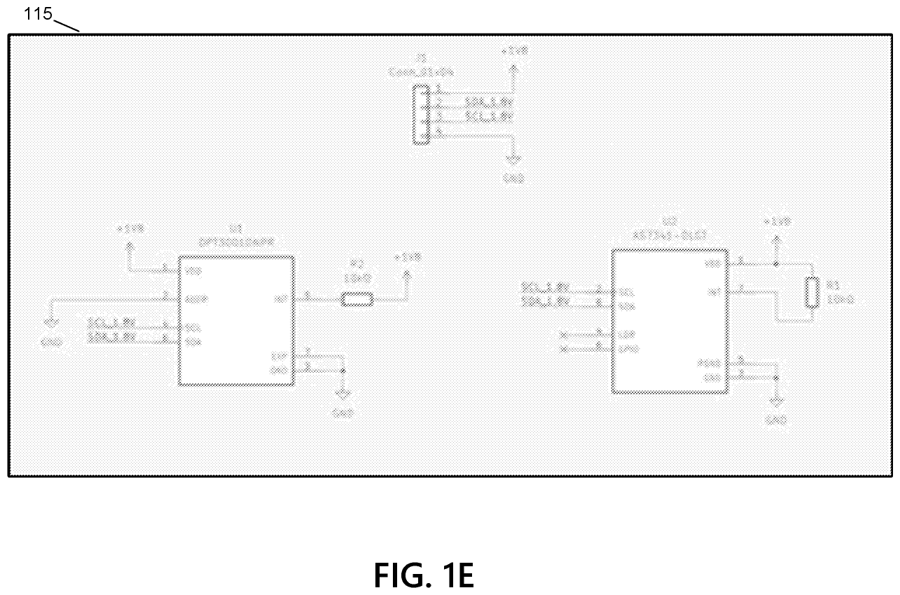

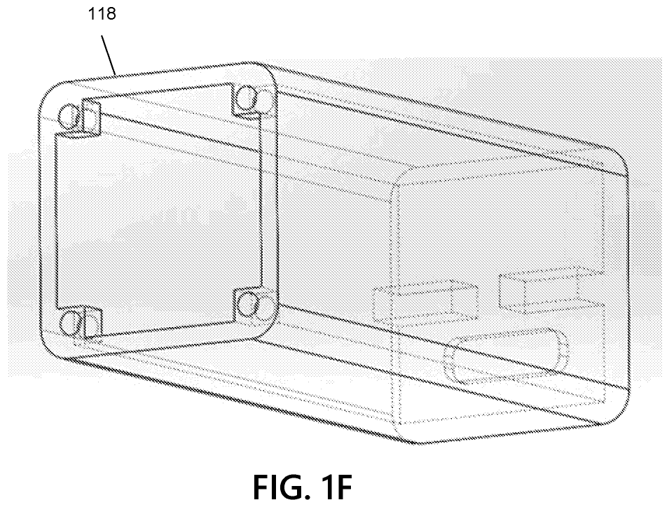

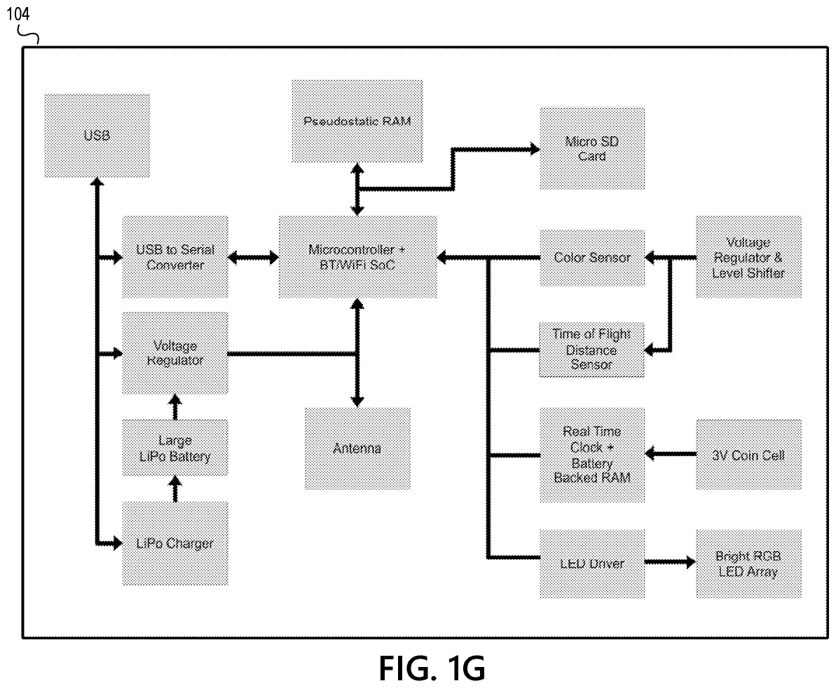

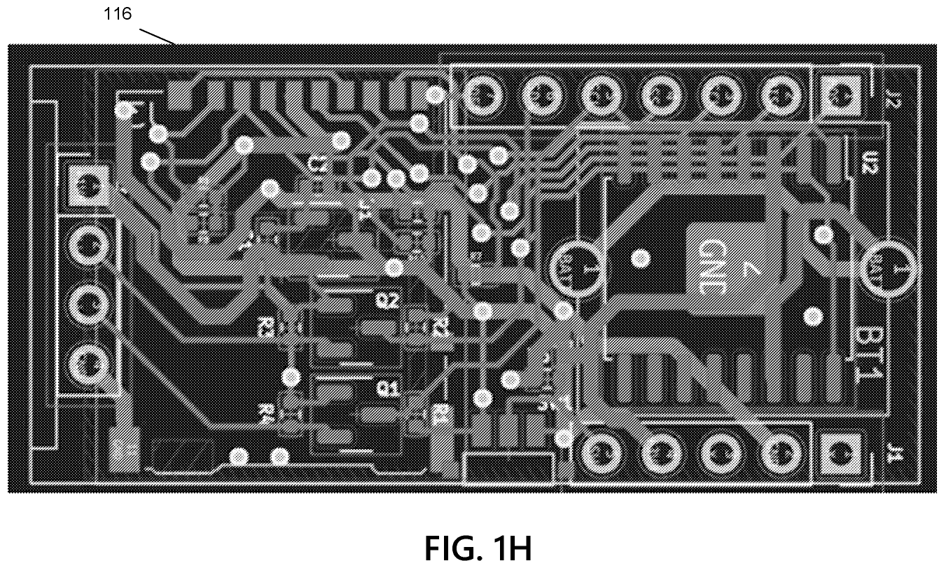

FIGS. 1A-1I depict an illustrative computing environment for implementing light sensors, a circadian rhythm recommendation model, and an intelligent light box in accordance with one or more illustrative embodiments described herein. Referring to FIG. 1A, computing environment 100 may include one or more computer systems. For example, computing environment 100 may include a circadian rhythm recommendation platform 102, light detection sensor 103, intelligent light box device 104, user device 105, and biometric sensor 106.

As described further below, circadian rhythm recommendation platform 102 may be a computer system that includes one or more computing devices (e.g., servers, or the like) and/or other computer components (e.g., processors, memories, communication interfaces, and/or other components) that may be used to train, host, support, and/or otherwise implement the circadian rhythm recommendation model, as described further below. In some instances, the circadian rhythm recommendation platform 102 may be configured to intake data and/or other information from other devices (such as the light detection sensor 103, user device 105, biometric sensor 106, and/or other devices), which may be used to train and/or otherwise implement the circadian rhythm recommendation model. Based on an output of the circadian rhythm recommendation model, the circadian rhythm recommendation platform 102 may be configured to output a control signal to one or more other devices (such as the light detection sensor 103, intelligent light box device 104, and/or other devices), which may cause such devices to modify and/or otherwise adjust their operations based on the control signal. Although certain features/functions are described herein and below as being performed by the circadian rhythm recommendation platform 102, such features/functions may, in some instances, be performed at a different device (such as locally on the user device 105 via an application stored at the user device 105) without departing from the scope of the disclosure.

Light detection sensor 103 may be or include a sensor device configured for light detection. For example, the sensor device may include Bluetooth, WiFi (802.11) or other components with wireless functionality (and/or an antenna) for communicating with other computing devices (e.g., circadian rhythm recommendation platform 102, user device 105, intelligent light box device 104, and/or other devices) in order to control, configure, program, and/or otherwise update the light detection sensor 103, and/or to transfer data from the light detection sensor 103 to the other computing devices. In some instances, the light detection sensor 103 may include a Universal Serial Bus (USB) connector or other external data connection for communicating with the other computing devices, in order to control, configure, program, or update the light detection sensor 103, and/or to transfer data from the light detection sensor 103 to the other computing devices. The USB or external data connection may also be used to provide power for recharging any rechargeable battery in the sensor device and/or independently power the device itself. In some instances, the light detection sensor 103 may be larger or smaller depending on the size of a corresponding battery. The larger the storage capacity of the battery, the higher the sampling rate that the light detection sensor 103 may be configured to support.

In some instances, internal connections between the components of the light detection sensor 103 may be via a data bus or direct connections. In some instances, components of the light detection sensor 103 (e.g., memory, Bluetooth, WiFi, or the like) may be included with the microcontroller or processor in a system-on-a-chip configuration to reduce the size and/or weight of the light detection sensor 103.

An example configuration of the light detection sensor 103 is shown, for example, in FIG. 1C. Furthermore, schematic diagrams 114 and 115 (shown in FIGS. 1D and 1E, respectively) depict example electronic configurations of the light detection sensor 103. In some instances, the light detection sensor 103 may be embedded or otherwise enclosed within a housing (such as light detection sensor housing 118, which is illustrated in FIG. 1F).

In certain embodiments, the light detection sensor 103 may be worn by a user so that it may detect the light that the user is exposed to during the course of their daily routine. For example, the light detection sensor 103 may be worn close to the eyes of the user, as the amount of light to which the user's eyes are exposed may be more useful data for determining recommendations than light measurement on another part of the body (e.g., through a sensor worn on the wrist or belt). More specifically, the closer the light detection sensor 103 is to the user's eyes, the more consistent the light measurements taken by the light detection sensor 103 will be to the light impacting the eyes of the user. As light may be a significant data point for use in analyzing and improving circadian rhythm (as is described further below), it may be important to have light information that most closely represents the light to which the user's eyes are exposed. By improving the collection of this data, the light detection sensor 103 may facilitate the improved analysis of circadian rhythms, and more effectively/accurately provide recommendations/guidance therein. For example, the light detection sensor 103 and/or it's housing may be configured as a sticker (e.g., to be worn on user's glasses or face), mounted on glasses, integrated into earbuds or headphones, mounted on a hard hat or other safety gear, a pendant, a clip, or a headband. In other embodiments, the light detection sensor 103 may be positioned near where a user would be while accomplishing a particular task in order to be exposed to a similar amount of light as the user's eyes. For example, the light detection sensor 103 may be positioned within an airplane cockpit, within a vehicle, or in an operating room, so as to measure the light to which the pilot, driver, or medical personnel is exposed. Similar configurations may be used for personnel such as military, lifeguards, firemen, hospital workers, and/or other individuals.

Intelligent light box device 104 may be configured to administer light of an appropriate color and intensity to a user. For example, the intelligent light box device 104 may include a lighting element (e.g., an RGB LED array, or the like) that may be adjustable for intensity, color, wavelength, color temperature, and/or otherwise. In some instances, the intelligent light box device 104 may include a microcontroller or processor coupled to a memory (e.g., pseudostatic RAM, flash memory, or other nonvolatile memory) storing software and operating instructions for the microcontroller. The memory connected to the microcontroller may also be used for storing data. In some instances, the intelligent light box device 104 may include a removable memory device, such as a MicroSD card, for storing data. In some instances, the intelligent light box device 104 may include an ambient light sensor to detect environmental light conditions around the light box that may be used to adjust the lighting element setting to achieve the desired effect.

In some instances, the intelligent light box device 104 may include one or more power sources, such as a rechargeable lithium polymer or lithium ion battery, or a replaceable battery. Associated circuitry may connect the power source to the rest of the intelligent light box device 104, and, in the case where a rechargeable battery is used, appropriate charging circuitry may also be included. In some instances, the intelligent light box device 104 may include Bluetooth, WiFi (802.11) or other components with wireless functionality (and/or an antenna) for communicating with other devices (e.g., circadian rhythm recommendation platform 102, light detection sensor 103, user device 105, and/or other devices), in order to control, configure, program, or update the intelligent light box device 104, as well as transfer data from the intelligent light box device 104 to these other devices. In some instances, the intelligent light box device 104 may include a USB connector or other external data connection for communicating with these other devices, in order to configure, program, or update the intelligent light box device 104, as well as to transfer data from the intelligent light box device 104 to the other devices. The USB or external data connection may also be used to provide power for recharging any rechargeable battery in the intelligent light box device 104 and/or independently power the intelligent light box device 104 itself. In some instances, the intelligent light box device 104 may include a distance sensor to determine and record the distance and angle that the users' position relative to the intelligent light box device 104 and/or other lighting element, and/or determine if the user is physically in front of the intelligent light box device 104 when light is being provided. In some instances, the intelligent light box device 104 may support automatic shut-off functionality to save battery life.

In some instances, internal connections between the components of the intelligent light box device 104 may be via a data bus or direct connections. Optionally, components of the intelligent light box device 104 (e.g., memory, Bluetooth, WiFi, or the like) may be included with the microcontroller or processor in a system-on-a-chip configuration.

An example configuration of the intelligent light box device 104 is shown, for example, in FIG. 1G. Furthermore, schematic diagrams 116 and 117 (shown in FIGS. 1H and 1I, respectively) depict example electronic configurations of the intelligent light box device 104.

User device 105 may be and/or otherwise include a laptop computer, desktop computer, mobile device, tablet, smartphone, and/or other device that may be used by an individual to access a circadian rhythm improvement application (which may, e.g., be locally hosted and/or supported by a remote computing system (e.g., circadian rhythm recommendation platform 102). In some instances, user device 105 may be configured to display one or more user interfaces (e.g., application interfaces, recommendation interfaces, or the like).

Biometric sensor 106 may be or include a sensor device configured for biometric data (e.g., pulse, heartrate, electrocardiogram readings, movement during the day/while asleep, and/or other information, and/or other biometric information) collection. For example, the sensor device may include Bluetooth, WiFi (802.11) or other components with wireless functionality (and/or an antenna) for communicating with other computing devices (e.g., circadian rhythm recommendation platform 102, user device 105, intelligent light box device 104, and/or other devices) in order to control, configure, program, and/or otherwise update the biometric sensor 106, and/or to transfer data from the biometric sensor 106 to the other computing devices. In some instances, the biometric sensor 106 may include a USB connector or other external data connection for communicating with the other computing devices, in order to control, configure, program, or update the biometric sensor 106, and/or to transfer data from the biometric sensor 106 to the other computing devices. The USB or external data connection may also be used to provide power for recharging any rechargeable battery in the sensor device and/or independently power the device itself.

In some instances, the biometric sensor 106 may be and/or otherwise be integrated into a user device (e.g., user device 105). In these instances, health capabilities of the user device may be configured to provide additional and/or alternative biometric data (e.g., health or activity data such as pulse heartrate, electrocardiogram readings, movements during the day and while asleep, and/or otherwise). For example, a health, fitness, and/or other API of an operating system of the user device may be configured to collect and provide this information.

In some instances, internal connections between the components of the biometric sensor 106 may be via a data bus or direct connections. In some instances, components of the biometric sensor 106 (e.g., memory, Bluetooth, WiFi, or the like) may be included with the microcontroller or processor in a system-on-a-chip configuration to reduce the size of the biometric sensor 106.

Computing environment 100 also may include one or more networks, which may interconnect circadian rhythm recommendation platform 102, light detection sensor 103, intelligent light box device 104, user device 105, and biometric sensor 106. For example, computing environment 100 may include a network 101 (which may interconnect, e.g., circadian rhythm recommendation platform 102, light detection sensor 103, intelligent light box device 104, user device 105, and biometric sensor 106).

In one or more arrangements, circadian rhythm recommendation platform 102, light detection sensor 103, intelligent light box device 104, user device 105, and biometric sensor 106 may be any type of computing device. For example, circadian rhythm recommendation platform 102, light detection sensor 103, intelligent light box device 104, user device 105, and biometric sensor 106 may, in some instances, be and/or include server computers, desktop computers, laptop computers, tablet computers, smart phones, or the like that may include one or more processors, memories, communication interfaces, storage devices, and/or other components. As noted above, and as illustrated in greater detail below, any and/or all of circadian rhythm recommendation platform 102, light detection sensor 103, intelligent light box device 104, user device 105, and/or biometric sensor 106 may be special-purpose computing devices configured to perform special functions.

Although the illustrative figure depicts a single light detection sensor 103, intelligent light box device 104, user device 105, and biometric sensor 106, any number of these devices may be implemented in the methods described herein without departing from the scope of the disclosure.

Referring to FIG. 1B, circadian rhythm recommendation platform 102 may include one or more processors 111, memory 112, and communication interface 113. A data bus may interconnect processor 111, memory 112, and communication interface 113. Communication interface 113 may be a network interface configured to support communication between circadian rhythm recommendation platform 102 and one or more networks (e.g., network 101, or the like). Memory 112 may include one or more program modules having instructions that, when executed by processor 111, cause circadian rhythm recommendation platform 102 to perform one or more functions described herein and/or one or more databases that may store and/or otherwise maintain information which may be used by such program modules and/or processor 111. In some instances, the one or more program modules and/or databases may be stored by and/or maintained in different memory units of circadian rhythm recommendation platform 102 and/or by different computing devices that may form and/or otherwise make up circadian rhythm recommendation platform 102. For example, memory 112 may have, host, store, and/or include circadian rhythm recommendation module 112a, circadian rhythm recommendation database 112b, and machine learning engine 112c.

Circadian rhythm recommendation module 112a may have instructions that direct and/or cause circadian rhythm recommendation platform 102 to provide improved techniques for providing recommendations to improve circadian rhythms, as discussed in greater detail below. Circadian rhythm recommendation database 112b may store information used by circadian rhythm recommendation module 112a and/or circadian rhythm recommendation platform 102 in application of advanced techniques to provide recommendations to improve circadian rhythms, and/or in performing other functions. Machine learning engine 112c may be used to dynamically train, host, and/or otherwise refine a machine learning model configured to produce circadian rhythm recommendations based on the input of user information, biometric data, light data, and/or other information.

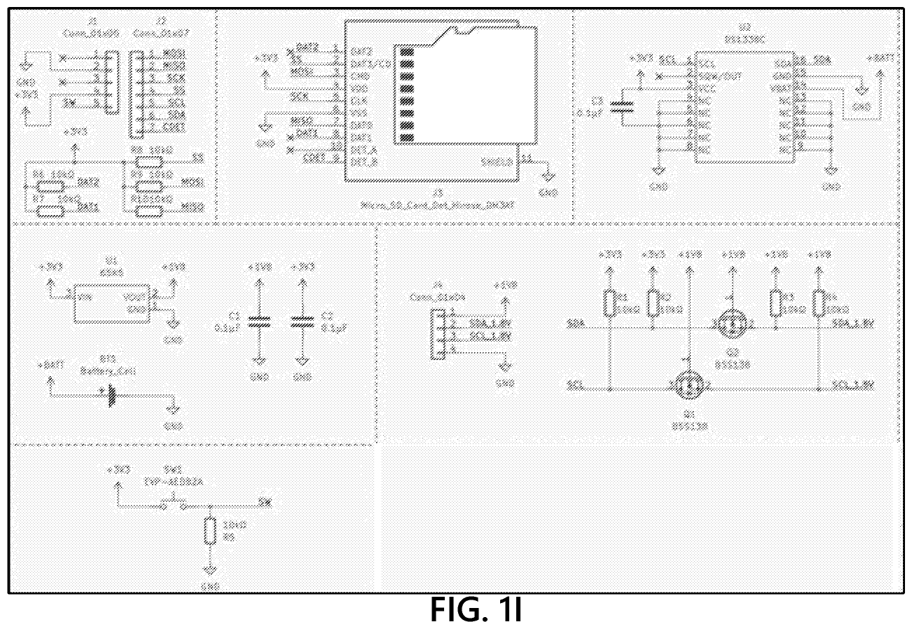

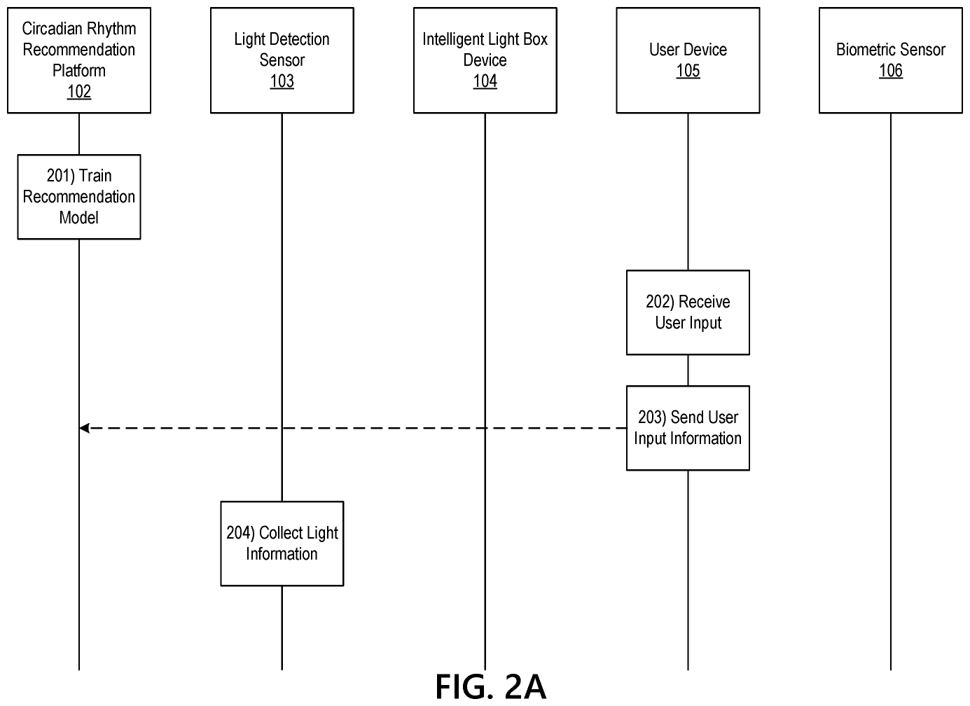

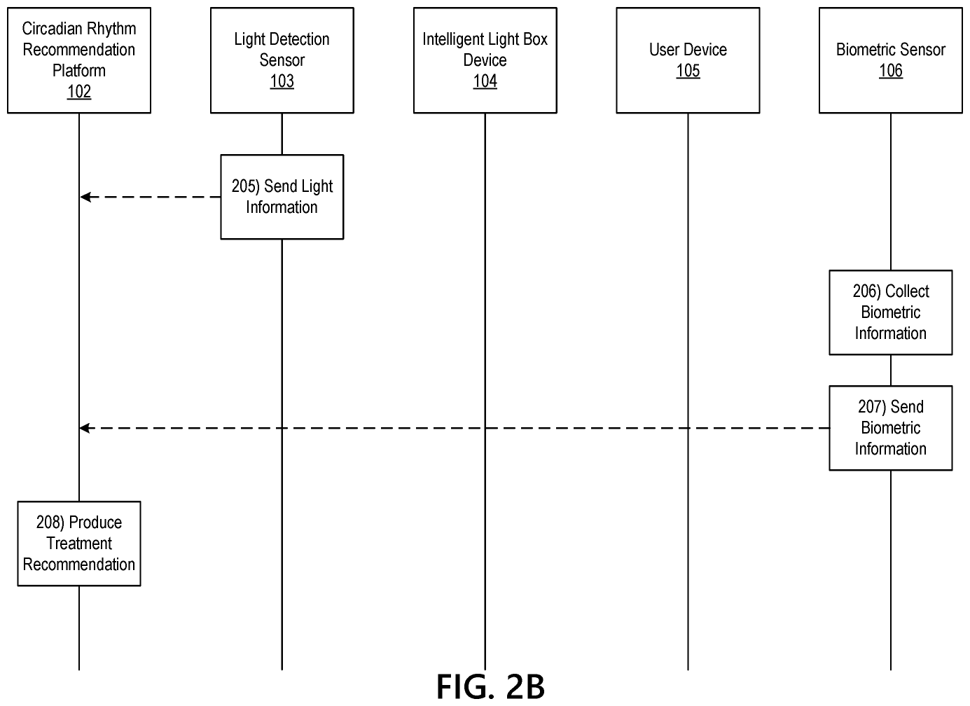

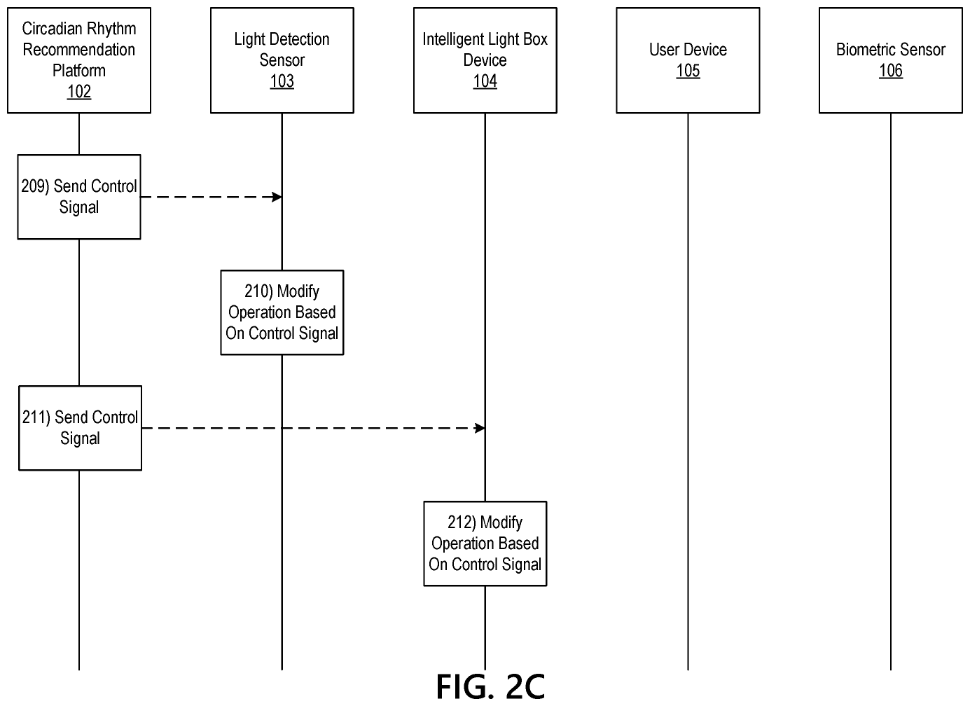

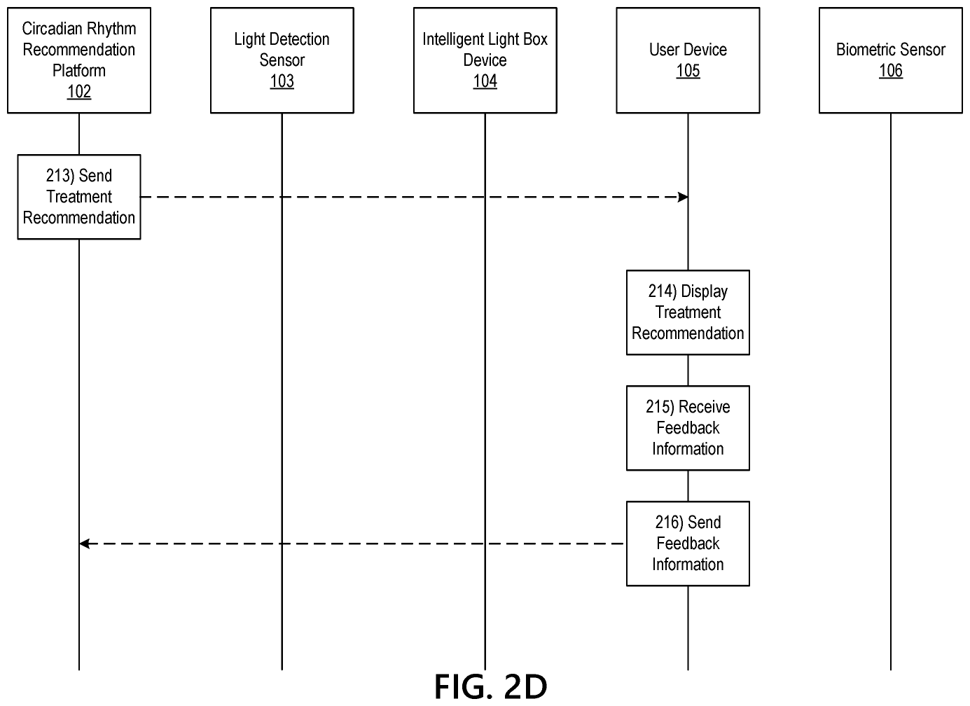

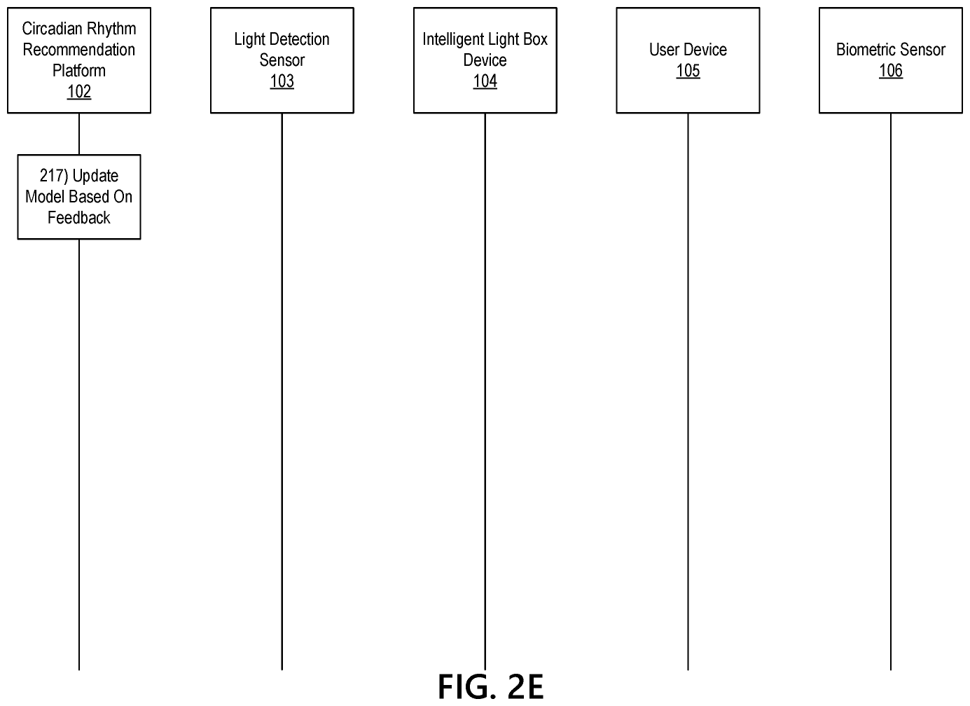

FIGS. 2A-2E depict an illustrative event sequence for implementing light sensors, a circadian rhythm recommendation model, and an intelligent light box in accordance with one or more example embodiments. Referring to FIG. 2A, at step 201, the circadian rhythm recommendation platform 102 may train a circadian rhythm recommendation model. For example, the circadian rhythm recommendation platform 102 may obtain historical information that may be used to train a machine learning and/or artificial intelligence model to output circadian rhythm recommendations (e.g., indicating steps that may be performed to improve circadian rhythm) and/or control signals (e.g., to cause performance of various actions at other devices such as the light detection sensor 103, intelligent light box device 104, and/or other devices). In some instances, the circadian rhythm recommendation platform 102 may train the machine learning model by inputting historical data from a light sensor (e.g., light detection sensor 103 and/or other sensors). Additionally or alternatively, the circadian rhythm recommendation platform may obtain historical light information (which may then be used to train the machine learning model) based on physical location information, such as Global Positioning System (GPS), weather, and/or other information, which may be indicative of light exposure (e.g., a user at a higher latitude or in cloudy weather conditions may be exposed to less natural sunlight than a user closer to the equator in sunny conditions). Additionally or alternatively, the circadian rhythm recommendation platform 102 may train the machine learning model by inputting historical data that may be self-reported by one or more users (e.g., using the user device 105 and/or other user devices), such as age, weight, gender, daily schedule, wake and sleep times, circadian phenotype (e.g., are you a morning person or an evening person), activity monitoring, menstrual cycle data, nutrition information, metabolic/metabolism data, cardio/pulmonary activity data, and/or other information. Additionally or alternatively, the circadian rhythm recommendation platform 102 may train the machine learning model by inputting historical data obtained from the user device 105, biometric sensor 106, and/or other devices such as health or activity data (e.g., pulse, heartrate, electrocardiogram readings, movement during the day/while asleep, and/or other information). For example, such information may be gathered by using an application programming interface (API) of a device operating system, such as a health API, fitness API, and/or other API.

In some instances, gathering of the historical information may include gathering data in a work environment (e.g., sensors worn on employee hardhats, sensors in a cockpit of a plane, sensors located within vehicles, sensors within a laboratory environment, sensors within an office environment, and/or other data) that may be used to predict employee fatigue. In these instances, work schedules of corresponding employees may be adjusted based on the collected information so as to staff employees on a given shift or at a given time to achieve maximum attentiveness and effectiveness. In some instances, gathering of this historical information may be performed using methods similar to the below described methods of gathering information about the user (e.g., with regard to the light detection sensor 103, user device 105, and biometric device 106).

Once collected, the circadian rhythm recommendation platform 102 may aggregate the data from a plurality of different users (from/about whom the historical information was received), so as to inform predictions for a particular user (e.g., the user described throughout and further below). For example, the circadian rhythm recommendation platform 102 may aggregate data from multiple users for research purposes and to refine recommendations produced by the below described machine learning model so as to provide better results based on individual user characteristics.

The circadian rhythm recommendation platform 102 may input this historical information into the machine learning model so as to train the machine learning model to establish correlations between particular habits and circadian rhythm. In doing so, the machine learning model may be trained to identify actions and/or recommendations for a given individual, that may be useful in improving that individual's circadian rhythm. In some instances, the circadian rhythm recommendation platform 102 may train a supervised machine learning model (e.g., artificial neural network, boosting, decision tree, nearest neighbor, support vector machine, random forest, and/or other supervised learning models), an unsupervised machine learning model (e.g., clustering, anomaly detection, and/or other unsupervised learning models), and/or other model.

Although use of the data aggregation is described further with regard to training the machine learning model to produce recommendation outputs and/or control signals, in some instances, such an aggregation of data may be used for other purposes without departing from the scope of the disclosure.

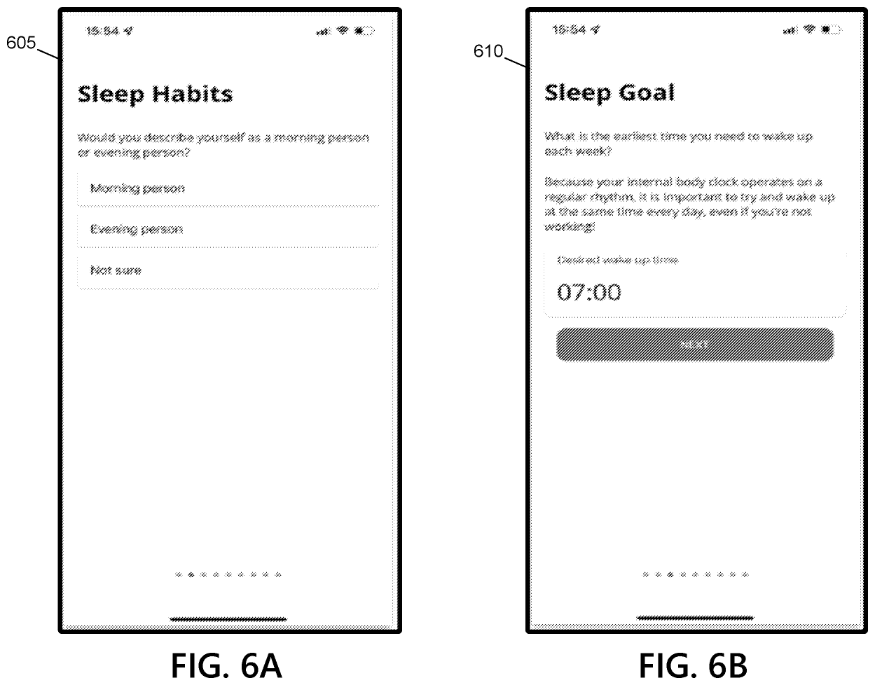

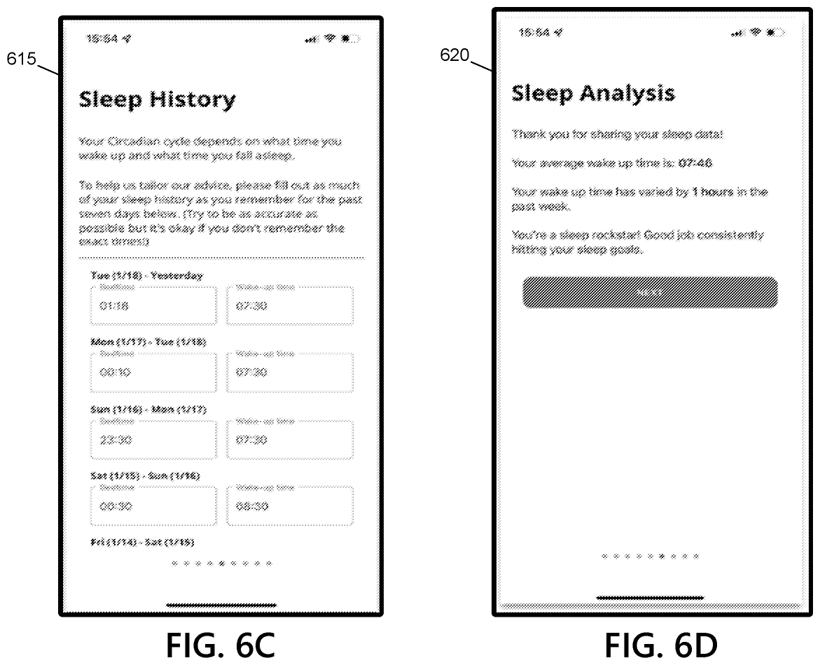

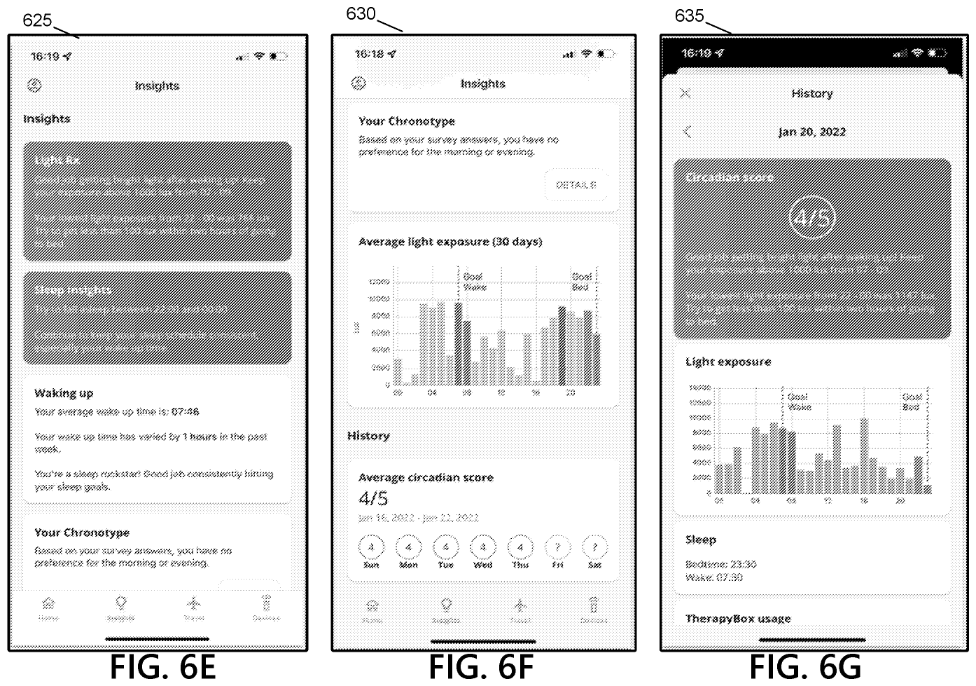

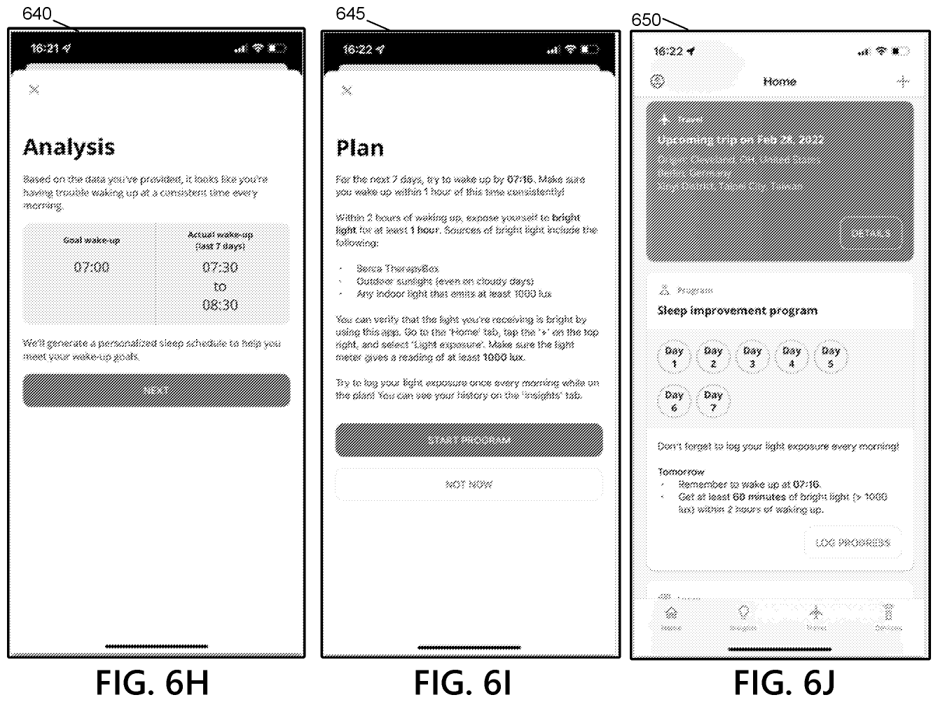

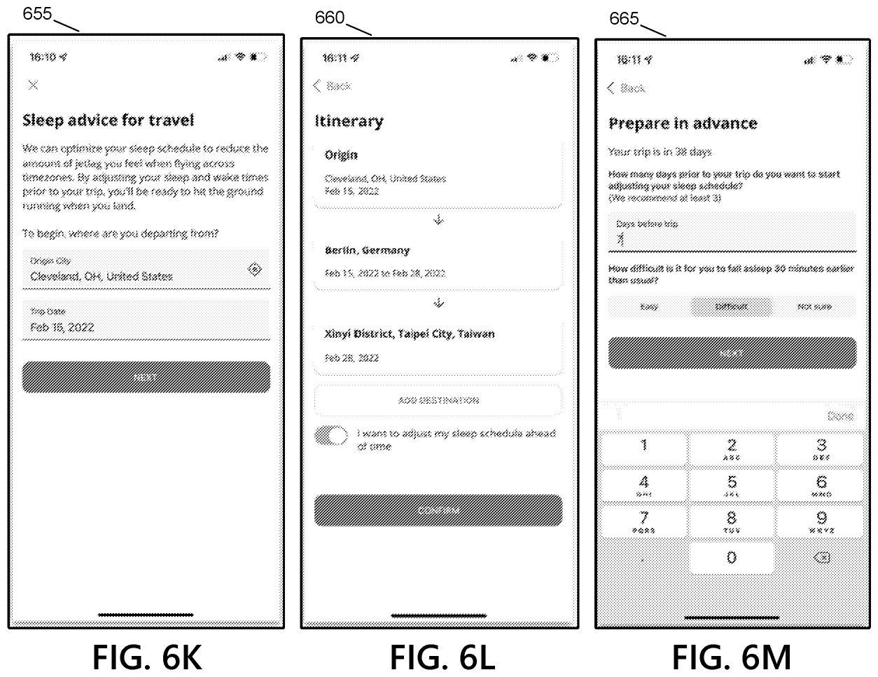

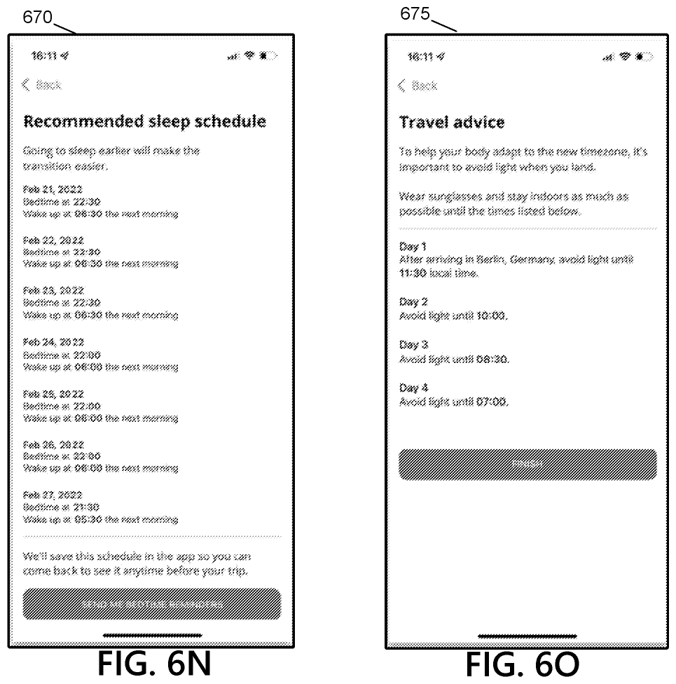

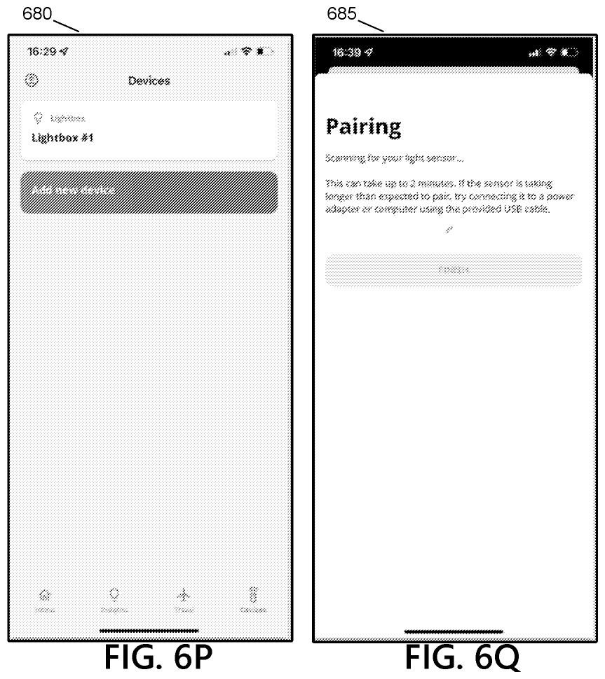

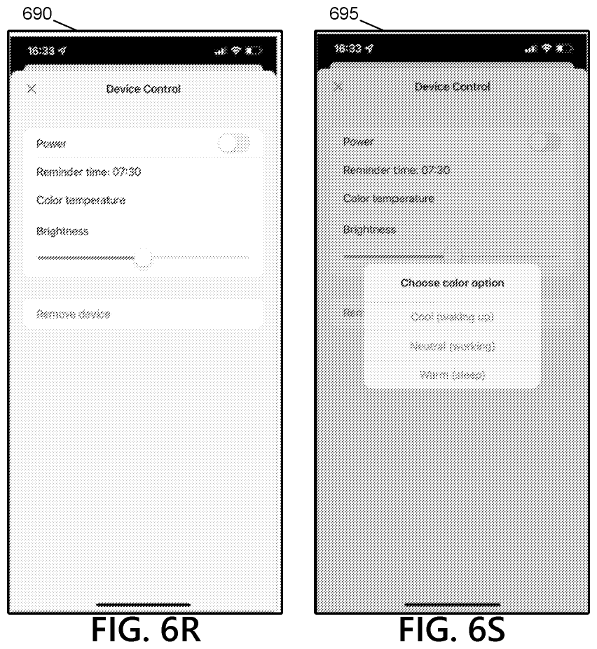

At step 202, the user device 105 may receive user input from a user. For example, the user device 105 may receive user input indicating age, weight, gender, daily schedule, wake and sleep times, circadian phenotype (e.g., are you a morning person or an evening person), activity monitoring, menstrual cycle data, nutrition information, metabolic/metabolism data, cardio/pulmonary activity data, location or GPS information, weather information, and/or other information. In some instances, along with providing this information about themselves, the user may provide information about desired goals (e.g., desired wake time, gross shifts to work time like overnight shifts at a hospital or manufacturing plant, and/or other goal information). In some instances, the user device 105 may receive this user input via a display of the user device 105 and via a graphical user interface, which may, e.g., be provided by an application (such as a circadian rhythm recommendation application, which may, e.g., be executed on the user device 105 and/or otherwise supported by the circadian rhythm recommendation platform 102). For example, the user device 105 may present a graphical user interface similar to graphical user interface 605 (which is illustrated in FIG. 6A), which may prompt the user to input their circadian phenotype. Additionally or alternatively, the user device 105 may present a graphical user interface similar to graphical user interface 610 (which is illustrated in FIG. 6B), which may prompt the user to input their desired wake up time. Additionally or alternatively, the user device 105 may present a graphical user interface similar to graphical user interface 615 (which is illustrated in FIG. 6C), which may prompt the user to input a sleep history. In some instances, once the user device 105 has received the user input, the user device 105 may display a graphical user interface similar to graphical user interface 620, which is displayed in FIG. 6D. For example, the user device 105 may display a summary of the user input.

In some instances, these interfaces (and/or other similar interfaces) may be presented to the user so as to obtain the historical information (described above with regard to step 201). Alternatively, similar information may have been collected for other users at step 201, and this information may be initially collected for the user at step 202 as part of an onboarding process (e.g., the machine learning model may have been trained using data from the other users and without training data from the user themself).

At step 203, the user device 105 may send user input information (e.g., based on the user input received at step 202) to the circadian rhythm recommendation platform 102. For example, the user device 105 may send the user input information while a wired and/or wireless connection is established with the circadian rhythm recommendation platform 102.

At step 204, the light detection sensor 103 may collect light information (e.g., light color, wavelength, intensity, type (e.g., artificial light, direct sunlight, indirect sunlight, or the like), and/or other information). For example, the light detection sensor 103 may be worn by the user so as to detect light that the user is exposed to during the course of their daily routine. Specifically, the light detection sensor 103 may be worn close to the eyes of the user, as the amount of light to which the user's eyes are exposed may be more useful data for determining recommendations than light measurement on another part of the body (e.g., through a sensor worn on the wrist or belt). For example, the light detection sensor 103 may be integrated into a pair of eyeglasses, headphones, earbuds, attached to a hat or headband, and/or otherwise positioned near the user's eyes.

In operation, the microcontroller of the light detection sensor 103 may periodically poll its light detection sensor to measure environmental light conditions. In between polling its light detection sensor, the light detection sensor 103 may enter a sleep state to extend battery life. The length of the polling and sleep intervals may be configured as desired to balance battery life and measurement frequency. For example, longer polling intervals may increase battery life, but may risk decreasing the accuracy of the light sensor data if, for example, the user is in a location where lighting conditions change frequently, or if the user is moving through various environments with different lighting conditions. In some instances, the light detection sensor 103 may be configured with a polling interval sufficiently short that the microcontroller may never enter a sleep state (though this may risk shortening battery life). In some instances, the polling interval may be provided by the clock in the microcontroller or an independently powered real-time clock component, for example, to reduce power consumption and prevent loss of time measurement in the event of depletion of the main rechargeable battery.

In some instances, the light detection sensor 103 may adjust the sampling based on how often the user is wearing the light detection sensor 103 and/or a relative importance of the situation. For example, if the user is only wearing the light detection sensor 103 for a short period of time, sampling may be higher so as to obtain enough data to enable accurate analysis/prediction (e.g., in comparison to a longer period of time, where the same number of data samples may be collected over a longer period of time, and thus a lower sampling rate may be implemented). Similarly, higher sampling rates may lead to higher accuracy of the collected data, and thus the more important the situation is, the higher the sampling rate may be and vice versa (e.g., higher sampling rate in an emergency room than sitting in a standard office, or the like).

In some instances, the light detection sensor 103 may collect light information only when worn by the user, which may be on a selective basis. For example, the user may be prompted (e.g., through a registration or training mode) to wear the light detection sensor 103 only at certain times or under certain conditions, which may enable the light detection sensor 103 to learn typical conditions for the user (e.g., the light in the user's office may be substantially the same throughout each day, so the user might not need to wear the light detection sensor 103 at all times once these conditions are recorded by the system). In some instances, this may take into account changes due to weather and/or seasonal changes, which may, e.g., have an effect on the user's daily light exposure.

In some instances, in collecting the light information for the user, the light detection sensor 103 may measure light intensity across all of the visible spectrum (i.e., from about 380 to about 750 nanometers). In some instances, the light detection sensor 103 may also capture data on exposure outside of the visible spectrum, such as infrared or ultraviolet light. In some instances, the light detection sensor 103 may measure a narrower range of the visible spectrum, such as about 400 to about 525 nanometers, or about 460 to about 489 nanometers, as these ranges may have a more significant influence on circadian rhythms.

In some instances, once the light information is collected, the light detection sensor 103 may store the light information locally in memory of the light detection sensor 103 or on a removeable memory card. In some instances, in storing the light information, the light detection sensor 103 may also store data including the date, time, and/or location at which each light sensor measurement was taken.

Referring to FIG. 2B, at step 205, the light detection sensor 103 may send the light information (collected at step 204) to the circadian rhythm recommendation platform 102. For example, the light detection sensor 103 may send the light information when a wired or wireless data connection is established between the light detection sensor 103 and the circadian rhythm recommendation platform 102. In some instances, the light information may be periodically and automatically transferred to the circadian rhythm recommendation platform 102. Alternatively, the user may manually transfer the light information using a removeable memory card or external data connection on the light detection sensor (e.g., when the light detection sensor is placed in a charging cradle).

In some instances, in addition or as an alternative to sending the light information, the light detection sensor 103 may send information about its status, such as battery level, charging status, WiFi/Bluetooth signal quality, diagnostic information about its hardware and software components, and/or other status information.

Although collection and transmission of the light information is described above with regard to the light detection sensor 103, such information may, in some instances, be provided using a camera (which may, e.g., be integrated into the user device 105 and/or other device), which may be configured to estimate lux values and/or collect other information, without departing from the scope of the disclosure.

At step 206, the biometric sensor 106 may collect biometric information. For example, the biometric sensor 106 may collect pulse, heartrate, electrocardiogram readings, movement during the day/while asleep, and/or other information, and/or other biometric information.

At step 207, the biometric sensor 106 may send the biometric information (collected at step 206) to the circadian rhythm recommendation platform 102. For example, the biometric sensor 106 may send the biometric information to the circadian rhythm recommendation platform 102 when a wired or wireless connection is established with the circadian rhythm recommendation platform 102.

Although the above steps describe receipt of data from the light detection sensor 103, the user device 105, and the biometric sensor 106, this is for illustrative purposes only, and any number of additional sensors may be used to provide data that may be used to inform and/or otherwise train the machine learning model and/or provide input data for the machine learning model without departing from the scope of the disclosure. For example, the circadian rhythm recommendation platform 102 may receive data from ambient environment sensors, pollutant sensors, nitric oxide sensors, carbon monoxide/dioxide sensors, cardio health sensors, temperature sensors, humidity sensors, ambient light sensors, and/or other sensors.

In some instances, the circadian rhythm recommendation platform 102 may implement security and/or privacy controls to ensure that any of the received data is not accessible to unauthorized persons.

At step 208, the circadian rhythm recommendation platform 102 may feed the user input information (received at step 203), light information (received at step 205), and the biometric information (received at step 207) into the machine learning model to produce a treatment recommendation. For example, the circadian rhythm recommendation platform 102 may identify, based on the received information specific to the user and historical information corresponding to a plurality of additional users, treatments that may improve a current circadian rhythm of the user. For example, the circadian rhythm recommendation platform 102 may identify historical data, similar to that of the user, and identify corresponding actions that caused an improvement in circadian rhythms for the corresponding data. Such actions may then be output by the machine learning model as circadian rhythm recommendations.

For example, based on the gathered data as well as information about the user's desired goals (e.g., desired wake time, gross shifts to work time like overnight shifts at a hospital or manufacturing plant), the machine learning model may produce outputs (e.g., recommendations) for the user about their wake and sleep schedule (e.g., when they should try to fall asleep, when to begin reducing their light exposure before their intended sleep time, when they should try to wake up in the morning, or the like). Additionally or alternatively, the machine learning model may also provide recommendations about the user's daily schedule including, for example, when to exercise, when to eat meals, when to administer circadian active compounds (e.g., melatonin), when to limit drug or alcohol intake, or the like. Additionally or alternatively, the machine learning model may provide recommendations on increasing light exposure including, for example, that the user should be exposed to certain colors and intensities of light at particular times of day. The machine learning model's recommendations may also be adjusted based on what the user's daily schedule allows. For example, light exposure in the morning may be the most effective treatment in adjusting a user's circadian rhythm, while administering melatonin in the evening may be less effective. However, if the user indicates that light exposure in the morning is inconvenient or impossible (e.g., in the case of a user whose sleeping quarters are shared with others), the machine learning model may adjust its recommendations in view of the user's limitations.

For a user with a stable schedule, the machine learning model may operate in an iterative fashion, adjusting recommendations over time and based on feedback from the user (e.g., manually entered feedback as well as data gathered from the user's devices) to better align the user's circadian rhythm with their daily schedule of activities.

In some instances, the machine learning model may also provide recommendations when the user anticipates that their schedule will change, for example, due to travel to another time zone or a change in the user's work schedule. In these instances, the machine learning model may include a desired shift as part of its determination of what recommendations to make for the user. This desired shift may include information about the change in the user's schedule and the date on which that change is expected to occur. For example, if the user is traveling to a destination in a different time zone, the desired shift might be from United States Eastern time to Paris, France time (i.e., six time zones advanced) occurring in three days. Using this desired shift information as well as the information it has about the user's current schedule and circadian rhythm, the machine learning model may then provide recommendations regarding, for example, sleep and wake times, and light exposure in advance of travel and after arrival at the destination to assist the user in adjusting to the new time zone. In some instances, such anticipated scheduling changes may have been manually input by the user and/or automatically detected (e.g., based on calendar information, or the like).

In these instances, the circadian rhythm recommendation platform 102 may communicate with the user device 105 to obtain additional details of the schedule variation (e.g., if not already received). For example, the circadian rhythm recommendation platform 102 may communicate with the user device 105 to display a recommended course of action to accommodate a variable or otherwise changing schedule (e.g., as shown in graphical user interface 650 of FIG. 6J). In these instances, the user device 105 may prompt the user for additional information about their upcoming schedule variation, such as a trip. For example, the user device 105 may display a prompt for travel details and other adjustment information (e.g., as depicted in graphical user interfaces 655-665, illustrated in FIGS. 6K-6M, respectively). The machine learning model may then identify recommendations to prepare the user in advance for adjusting to the time change, such as recommended sleep schedules and/or travel advice (e.g., as are described further below with regard to step 214).

Referring to FIG. 2C, at step 209, the circadian rhythm recommendation platform 102 may generate and send a first control signal, based on the output produced by the machine learning model at step 208, to the light detection sensor 103. For example, the circadian rhythm recommendation platform 102 may send the first control signal while a wired or wireless data connection is established with the light detection sensor 103. For example, the first control signal may direct the light detection sensor 103 to set and/or adjust the polling and/or sleep intervals for the light detection sensor 103, the frequency at which the light detection sensor 103 transmits data to the machine learning model, and/or other control information.

At step 210, based on or in response to the first control signal sent at step 209, the light detection sensor 103 may modify its operation. For example, the light detection sensor 103 may set and/or adjust the polling and/or sleep intervals, the data transmission frequency, and/or other operational characteristics.

At step 211, the circadian rhythm recommendation platform 102 may generate and send a second control signal, based on the output produced by the machine learning model at step 208, to the intelligent light box device 104. For example, the circadian rhythm recommendation platform 102 may send the second control signal while a wired or wireless connection is established with the intelligent light box device 104 (e.g., Bluetooth, WiFi (802.11), or the like). For example, the second control signal may indicate a frequency at which the intelligent light box device 104 should transmit data to the machine learning model and/or other control information for configuring the light box (e.g., settings information such as intensity and/or color of light, time and duration of use, and/or other information).

At step 212, based on or in response to the second control signal (sent at step 211), the intelligent light box device 104 may modify its operation. For example, the intelligent light box device 104 may adjust a frequency of data transmission and/or other settings information (e.g., intensity and/or color of light, time and duration of use, and/or other information). Additionally or alternatively, such actions by the intelligent light box device 104 may be made using physical controls (e.g., buttons or knobs) on the device itself and/or via manual control through an application executed at the user device 105 (e.g., as depicted in graphical user interface 690 of FIG. 6R and graphical user interface 695 of FIG. 6S, or the like).

After adjusting based on the second control signal, the intelligent light box device 104 may be configured for use (e.g., by the user) based on the output produced by the machine learning model. For example, the intelligent light box device 104 may emit light of a given intensity and/or color for a given time/duration (e.g., which may have been specified as an output of the machine learning model). In some instances, during its use, the intelligent light box device 104 may transmit data back to the machine learning model (e.g., intensity, color, user position, time, duration of use, battery level, charging status, diagnostic information about hardware and software components, and/or other data). In these instances, the circadian rhythm recommendation platform 102 and/or machine learning model may dynamically adjust settings for the intelligent light box device 104 based on receipt of the feedback data so as to continually refine and improve operation of the intelligent light box device 104 over time.

In some instances, the intelligent light box device 104 may be configured to dynamically communicate with the light detection sensor 103 and to automatically adjust its settings (e.g., intensity, color, or the like) based on information from the light detection sensor 103. For example, the second control signal and/or another signal may have directed the intelligent light box device 104 to output a light of a particular intensity. However, based on light information from the light detection sensor 103 (which may, e.g., be worn by the user during their use of the intelligent light box device 104), the intelligent light box device 104 may identify that due to a distance between the user and the intelligent light box device 104, the output intensity must be adjusted so as to provide the proper intensity to the user (e.g., the intensity experienced at the user may reduce as the distance from the light box increases). Accordingly, the intelligent light box device 104 may automatically adjust the intensity of the output. As a particular example, the second control signal may direct the intelligent light box device 104 to output 1500 lux with the intention of causing roughly 1200 lux to be observed or experienced by the user (e.g., based on reduction in the lux due to a distance between the intelligent light box device 104 and the user). If the light detection sensor 103, however, detects that the actual received light intensity is higher or lower than 1200 lux, it may direct the intelligent light box device 104 to adjust accordingly (which may, e.g., cause the intelligent light box device 104 to vary the light output accordingly).

Similar adjustments may be made by the intelligent light box device 104 without the use of the light detection sensor 103. For example, the intelligent light box device 104 may be configured with a distance sensor, capable of measuring distance between the user and the intelligent light box device 104. In these instances, the intelligent light box device 104 may adjust the output intensity based on the identified distance so as to cause a correct intensity to be observed by the user (e.g., increase intensity of the output as distance increases and vice versa).

Accordingly, a closed loop system between the circadian rhythm recommendation platform 102, light detection sensor 103, intelligent light box device 104, and/or user device 105 may be described, in which data from the light detection sensor 103 and/or user device 105 may be used to inform a model hosted by the circadian rhythm recommendation platform 102/user device 105, which in turn may output recommendations and/or control signals which may be sent and used to control the user device 105 and/or intelligent light box device 104 so as to provide overall improvements to a circadian rhythm of the user.

Referring to FIG. 2D, at step 213, the circadian rhythm recommendation platform 102 may send treatment recommendation information, based on the output produced by the machine learning model at step 208, to the user device 105. For example, the circadian rhythm recommendation platform 102 may send the treatment recommendation information when a wired or wireless connection is established with the user device 105. In some instances, the circadian rhythm recommendation platform 102 may also send one or more commands directing the user device 105 to display the treatment recommendation information.

At step 214, based on or in response to the one or more commands directing the user device 105 to display the treatment recommendation information, the user device 105 may display the treatment recommendation information. In some instances, the treatment recommendation information may include analysis of the user's current information, such as insights associated with the users wake up times, sleep, chronotype, light exposure, circadian score, and/or other information (e.g., as depicted in graphical user interfaces 625-635 of FIG. 6E-6G, respectively). For example, the user device 105 may display a graphical user interface similar to graphical user interface 640, which is shown in FIG. 6H, which may include an analysis of the users sleep habits. Additionally or alternatively, the user device 105 may display a recommended course of action, which may, e.g., include use of the intelligent light box device 104 (e.g., as shown in graphical user interface 645 of FIG. 6I). In these instances, the user device 105 may display graphical user interfaces to pair the intelligent light box device 104 with the user device 105 (e.g., graphical user interface 680 of FIG. 6P, graphical user interface 685 of FIG. 6Q, and/or otherwise).

Additionally or alternatively, the user device 105 may display a recommended course of action to accommodate a variable or otherwise changing schedule (e.g., as shown in graphical user interface 650 of FIG. 6J). For example, the user device 105 may display a recommended sleep schedule (e.g., as shown in graphical user interface 670 of FIG. 6N), travel advice (e.g., as shown in graphical user interface 675 of FIG. 6O), and/or other information.

At step 215, the user device 105 may receive feedback information from the user (e.g., via a graphical user interface displayed at the user device 105). For example, the user may provide feedback to indicate whether or not they are fulfilling the recommendations and/or to measure if the recommendations are improving the user's circadian rhythm. Additionally or alternatively, feedback information may be provided by other devices (e.g., the biometric sensor 106, the light detection sensor 103, and/or other devices) to automatically measure whether the user's circadian rhythm is improving. This on-going monitoring may be used to refine and/or otherwise adjust the machine learning model so as to dynamically improve future recommendations provided to the user (e.g., to increase the likelihood that future recommendations may cause an improvement to the user's circadian rhythm).

At step 216, the user device 105 (and/or other devices) may send the feedback information (received at step 215) to the circadian rhythm recommendation platform 102. For example, the user device 105 may send the feedback information when a wired or wireless data connection is established with the circadian rhythm recommendation platform 102.

Referring to FIG. 2E, at step 217, the circadian rhythm recommendation platform 102 may refine and/or otherwise adjust the machine learning model based on the feedback information so as to dynamically improve the accuracy of the machine learning model in producing future recommendations provided to the user (e.g., to increase the likelihood that future recommendations may cause an improvement to the user's circadian rhythm).

Although the illustrative event sequence describes use of the machine learning model hosted at the circadian rhythm recommendation platform 102, such a model may, in some instances, be hosted in an application executed directly on the user device 105 without departing from the scope of the disclosure. Furthermore, although the steps are shown in a specific order, these steps may occur in alternative or different orders without departing from the scope of the disclosure. For example, light information, user input information, and/or biometric information may be collected in a different order without departing from the scope of the disclosure. Similarly, the control signals and/or recommendation information may be sent to the light detection sensor 103, intelligent light box device 104, and/or user device 105 without departing from the scope of the disclosure.

Furthermore, although the illustrative event sequence primarily describes optimization of one's own circadian rhythm, the systems and methods described herein may be used to achieve additional or alternative outputs/goals without departing from the scope of the disclosure. For example, the methods may be used to optimize an individual's circadian rhythm to better achieve weight loss, treat medical conditions (e.g., diabetes treatment, cancer treatment, infectious diseases, respiratory conditions, cardiac conditions, coronary artery disease, pain, and/or other conditions), identify optimal work schedules, and/or for other use cases. For example, in the medical treatment context, the methods described herein may be used to identify an optimal (e.g., most effective in terms of treating or otherwise preventing the corresponding condition) time for administering a treatment, a dosage for the treatment, and/or to produce other insights.

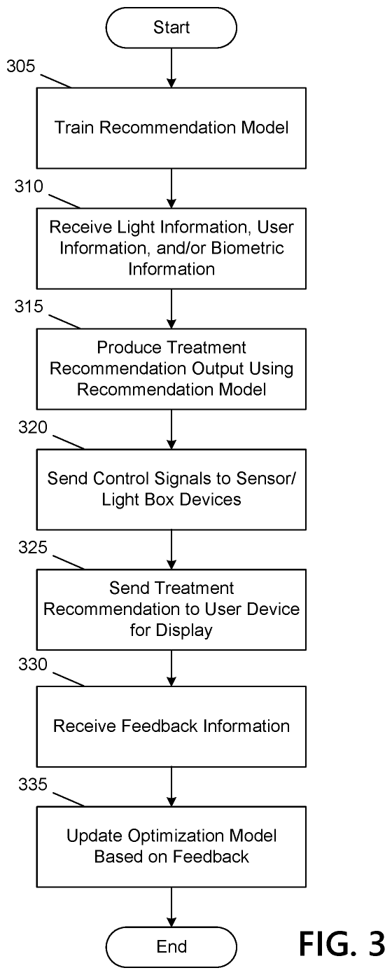

FIG. 3 depicts an illustrative method for implementing a circadian rhythm recommendation model in accordance with one or more example embodiments. At step 305, a computing platform (e.g., the circadian rhythm recommendation platform 102) may train a machine learning model to output recommendations to improve circadian rhythm. At step 310, the computing platform may receive light information, user input information, and/or biometric information for a user. At step 315, the computing platform may input the light information, the user input information, and/or the biometric information into the machine learning model to produce a treatment recommendation output. At step 320, the computing platform may send control signals to sensors (e.g., light detection sensor 103) and/or intelligent light box devices (e.g., intelligent light box device 104). At step 325, the computing platform may send a treatment recommendation to a user device (e.g., user device 105) for display. At step 330, the computing platform may receive feedback information from the user device and/or other devices. At step 335, the computing platform may update the machine learning model based on the feedback information.

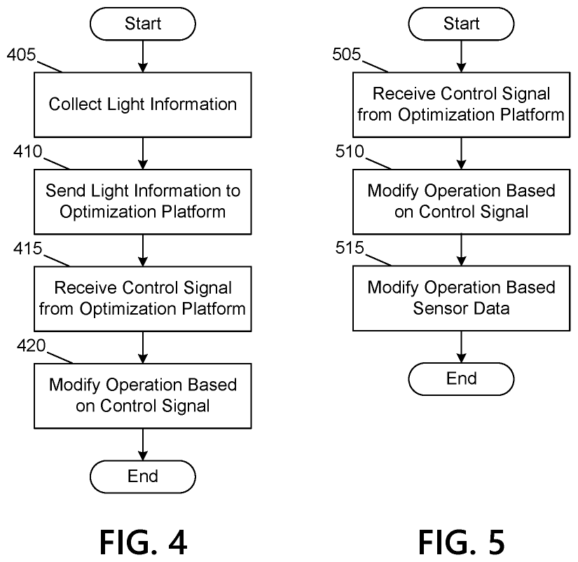

FIG. 4 depicts an illustrative method for implementing a light sensor in accordance with one or more example embodiments. At step 405, a computing platform (e.g., the light detection sensor 103) may collect light information. At step 410, the computing platform may send the light information to an optimization platform (e.g., the circadian rhythm recommendation platform 102). At step 415, the computing platform may receive a control signal from the optimization platform. At step 420, the computing platform may modify its operation based on the control signal.

FIG. 5 depicts an illustrative method for implementing an intelligent light box device in accordance with one or more example embodiments. At step 505, the intelligent light box device (e.g., intelligent light box device 104) may receive a control signal from an optimization platform (e.g., the circadian rhythm recommendation platform 102). At step 510, the intelligent light box device may modify its operation based on the control signal. At 515, the intelligent light box device may modify its operation based on data collected from sensors integrated into and/or otherwise associated with the intelligent light box device itself (e.g., ambient light sensors, distance sensors, or the like).

One or more aspects of the disclosure may be embodied in computer-usable data or computer-executable instructions, such as in one or more program modules, executed by one or more computers or other devices to perform the operations described herein. Generally, program modules include routines, programs, objects, components, data structures, and the like that perform particular tasks or implement particular abstract data types when executed by one or more processors in a computer or other data processing device. The computer-executable instructions may be stored as computer-readable instructions on a computer-readable medium such as a hard disk, optical disk, removable storage media, solid-state memory, RAM, and the like. The functionality of the program modules may be combined or distributed as desired in various embodiments. In addition, the functionality may be embodied in whole or in part in firmware or hardware equivalents, such as integrated circuits, application-specific integrated circuits (ASICs), field programmable gate arrays (FPGA), and the like. Particular data structures may be used to more effectively implement one or more aspects of the disclosure, and such data structures are contemplated to be within the scope of computer executable instructions and computer-usable data described herein.

Various aspects described herein may be embodied as a method, an apparatus, or as one or more computer-readable media storing computer-executable instructions. Accordingly, those aspects may take the form of an entirely hardware embodiment, an entirely software embodiment, an entirely firmware embodiment, or an embodiment combining software, hardware, and firmware aspects in any combination. In addition, various signals representing data or events as described herein may be transferred between a source and a destination in the form of light or electromagnetic waves traveling through signal-conducting media such as metal wires, optical fibers, or wireless transmission media (e.g., air or space). In general, the one or more computer-readable media may be and/or include one or more non-transitory computer-readable media.

As described herein, the various methods and acts may be operative across one or more computing servers and one or more networks. The functionality may be distributed in any manner, or may be located in a single computing device (e.g., a server, a client computer, and the like). For example, in alternative embodiments, one or more of the computing platforms discussed above may be combined into a single computing platform, and the various functions of each computing platform may be performed by the single computing platform. In such arrangements, any and/or all of the above-discussed communications between computing platforms may correspond to data being accessed, moved, modified, updated, and/or otherwise used by the single computing platform. Additionally or alternatively, one or more of the computing platforms discussed above may be implemented in one or more virtual machines that are provided by one or more physical computing devices. In such arrangements, the various functions of each computing platform may be performed by the one or more virtual machines, and any and/or all of the above-discussed communications between computing platforms may correspond to data being accessed, moved, modified, updated, and/or otherwise used by the one or more virtual machines.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps depicted in the illustrative figures may be performed in other than the recited order, and one or more depicted steps may be optional in accordance with aspects of the disclosure.

What is claimed is:

1. A computing platform comprising:
at least one processor;
a communication interface communicatively coupled to the at least one processor; and
memory storing computer-readable instructions that, when executed by the at least one processor, cause the computing platform to:
receive, from a light sensor device via the communication interface, light information;
receive, from a user device via the communication interface, user information and desired outcome information;
input, into a circadian rhythm recommendation model, the light information, the desired outcome information, and the user information, wherein inputting the light information, the desired outcome information, and the user information into the circadian rhythm recommendation model causes the circadian rhythm recommendation model to produce a first circadian rhythm improvement recommendation;
send, to the user device, the first circadian rhythm improvement recommendation and one or more commands directing the user device to display the first circadian rhythm improvement recommendation, wherein sending the one or more commands directing the user device to display the first circadian rhythm improvement recommendation causes the user device to display the first circadian rhythm improvement recommendation;
receive, from the user device, feedback information indicating compliance with the first circadian rhythm improvement recommendation; and
refine, using the feedback information, the circadian rhythm recommendation model.

2. The computing platform of claim 1, wherein the memory stores additional computer readable instructions that, when executed by the at least one processor, cause the computing platform to:
train, using historical information, the circadian rhythm recommendation model, wherein training the circadian rhythm recommendation model configures the circadian rhythm recommendation model to produce control signals and circadian rhythm improvement recommendations.

3. The computing platform of claim 2, wherein the historical information corresponds to a plurality of users.

4. The computing platform of claim 2, wherein the historical information comprises one or more of: historical light information, historical user information, or historical biometric information.

5. The computing platform of claim 1, wherein the light information comprises one or more of: light intensity across a visible spectrum or light intensity outside of the visible spectrum.

6. The computing platform of claim 1, wherein the user information comprises one or more of: age, weight, gender, daily schedule, wake and sleep times, circadian phenotype, activity information, menstrual cycle data, nutrition information, metabolic/metabolism data, cardio/pulmonary activity data, location information, or weather information.

7. The computing platform of claim 1, wherein the desired outcome information comprises one or more of: travel adjustments or schedule adjustments.

8. The computing platform of claim 1, wherein the memory stores additional computer readable instructions that, when executed by the at least one processor, further cause the computing platform to:
receive biometric information from a biometric sensor, wherein producing the first circadian rhythm improvement recommendation is further based on the biometric information.

9. The computing platform of claim 8, wherein the biometric information comprises one or more of: pulse information, heartrate information, electrocardiogram readings, or movement information.

10. The computing platform of claim 1, wherein the first circadian rhythm improvement recommendation includes one or more of: use of a light box, adjust sun exposure, adjust light exposure, a recommended wake up time, a recommended sleep schedule, or travel advice.

11. The computing platform of claim 1, wherein inputting the light information and the user information into the circadian rhythm recommendation model further causes the circadian rhythm recommendation model to produce a first control signal directed to an intelligent light box device.

12. The computing platform of claim 11, wherein the memory stores additional computer readable instructions that, when executed by the at least one processor, cause the computing platform to:
send, to the intelligent light box device, the first control signal, wherein sending the first control signal to the intelligent light box device causes the intelligent light box device to perform one or more of: adjust a frequency of data transmission, adjust an intensity of emitted light, adjust a color of the emitted light, adjust a time of use, or adjust a duration of use.

13. The computing platform of claim 11, wherein the light sensor device is configured to communicate an observed intensity of light to the intelligent light box device, and wherein the intelligent light box device is configured to automatically adjust an emitted intensity of light based on the observed intensity of light.

14. The computing platform of claim 1, wherein the memory stores additional computer readable instructions that, when executed by the at least one processor, further cause the computing platform to:
send, to the light sensor device, the first control signal, wherein sending the first control signal to the light sensor device causes the light sensor device to modify one or more operations of the light sensor device.

15. The computing platform of claim 14, wherein modifying the one or more operations of the light sensor device comprises one or more of: adjust polling intervals, adjust sleep intervals, or adjust data transmission frequency.

16. The computing platform of claim 1, wherein the light sensor device is mounted to a pair of glasses.

17. A method comprising:
at a computing platform comprising at least one processor, a communication interface, and memory:
receiving, from a light sensor device and via the communication interface, light information;
receiving, from a user device and via the communication interface, user information and desired outcome information;
inputting, into a circadian rhythm recommendation model, the light information, the desired outcome information, and the user information, wherein inputting the light information, the desired outcome information, and the user information into the circadian rhythm recommendation model causes the circadian rhythm recommendation model to produce a first circadian rhythm improvement recommendation;
sending, to the user device, the first circadian rhythm improvement recommendation and one or more commands directing the user device to display the first circadian rhythm improvement recommendation, wherein sending the one or more commands directing the user device to display the first circadian rhythm improvement recommendation causes the user device to display the first circadian rhythm improvement recommendation;
receiving, from the user device, feedback information indicating compliance with the first circadian rhythm improvement recommendation; and
refining, using the feedback information, the circadian rhythm recommendation model.

18. The method of claim 17, further comprising:
training, using historical information, the circadian rhythm recommendation model, wherein training the circadian rhythm recommendation model configures the circadian rhythm recommendation model to produce circadian rhythm improvement recommendations.

19. The method of claim 18, wherein the historical information corresponds to a plurality of users.

20. One or more non-transitory computer-readable media storing instructions that, when executed by a computing platform comprising at least one processor, a communication interface, and memory, cause the computing platform to:
receive, from a light sensor device and via the communication interface, light information;
receive, from a user device and via the communication interface, user information and desired outcome information;
input, into a circadian rhythm recommendation model, the light information, the desired outcome information, and the user information, wherein inputting the light information, the desired outcome information, and the user information into the circadian rhythm recommendation model causes the circadian rhythm recommendation model to produce a first circadian rhythm improvement recommendation;
send, to the user device, the first circadian rhythm improvement recommendation and one or more commands directing the user device to display the first circadian rhythm improvement recommendation, wherein sending the one or more commands directing the user device to display the first circadian rhythm improvement recommendation causes the user device to display the first circadian rhythm improvement recommendation;
receive, from the user device, feedback information indicating compliance with the first circadian rhythm improvement recommendation; and
refine, using the feedback information, the circadian rhythm recommendation model.
